SEQUENCE LISTING (4 sequences)

SEQ ID NO: 1 - protein, 323 aa, synthetic construct
  REGION 1..323: note = Partial Sequence of LRPAP1
YSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 2 - protein, 357 aa, synthetic construct
  REGION 1..357: note = Full length of LRPAP1
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 3 - protein, 324 aa, synthetic construct
  REGION 1..324: note = Truncated LRPAP1
MYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 4 - protein, 324 aa, synthetic construct
  REGION 1..324: note = RAPm6
MYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKFNFCQKQLEIAFEKLRHAESVGDGERVSRSREKFALLEGRCKELGYTVKKHLQDLSGRISRARHNEL

(19) United States
(12) Patent Application Publication
(10) Pub. No.: US 2024/0307489 A1
(43) Pub. Date: Sep. 19, 2024

(54) USE OF A LRP1 INHIBITOR IN TREATING NOTCH SIGNALING-DEPENDENT DISEASE

(71) Applicant: Westlake University, Hangzhou, Zhejiang (CN)

(72) Inventor: Xu Li, Hangzhou, Zhejiang (CN)

(21) Appl. No.: 18/576,006

(22) PCT Filed: Jul. 1, 2022
(86) PCT No.: PCT/CN2022/103347

§ 371 Date: Jan. 2, 2024

(30) Foreign Application Priority Data
Jul. 2, 2021 (WO) PCT/CN2021/104314

(51) Int. Cl. A61K 38/17; A61P 35/02; A61P 35/04; C12N 15/113; G01N 33/50; G01N 33/92

(52) U.S. Cl. CPC A61K 38/177; A61P 35/02; A61P 35/04; C12N 15/1138; G01N 33/5011; G01N 33/92; C12N 2310/14

(57) ABSTRACT

The present invention refers to a method for treating Notch signaling-dependent disease in the subject with a LRP1 specific inhibitor. The Notch signaling-dependent disease is selected from leukemia. Also provided is a method for screening a drug treating Notch signaling-dependent disease using LRP1 as a target.

### TECHNICAL FIELD

The present disclosure generally relates to a method for the down-regulation of LRP1 using related inhibitors so as to treat Notch signaling-dependent disease.

### BACKGROUND

Notch signaling is highly conserved in various species ranging from Caenorhabditis elegans (C. elegans) to mammals and is regarded as one of the most important signaling pathways. Dysregulation of Notch signaling has been linked to multiple human disorders ranging from developmental syndromes to complex diseases such as Alzheimer's disease, cardiovascular diseases, and cancers. Activating mutations of Notch 1/2 have been uncovered in patients with T-cell acute lymphoblastic leukemia (T-ALL), chronic lymphocytic leukemia (CLL), and many other types of cancers, while loss-of-function mutations in Notch receptors have been identified in patients with several squamous cell carcinomas (SCC).

LRP1 is a large multiligand endocytic receptor that belongs to the low-density lipoprotein receptor family. Members of this family have been reported to be involved in cholesterol metabolism, intracellular trafficking and cell signal transduction, as well as in the regulation of cell migration, proliferation, synaptic plasticity, neuron development and cerebral vascular permeability maintenance. LRP1 is synthesized as a 600-kDa precursor protein and then processed into an extracellular ligand-binding subunit of 515 kDa (LRP1α) and a transmembrane and intracellular subunit of 85 kDa (LRP1β), which is associated with efficient endocytic trafficking and intracellular signal transduction. As an endocytic receptor, LRP1 promotes the internalization of many extracellular ligands, such as PDGFR-β, amyloid-β, Tau and CCN2, through endocytosis and transfers them to endosomes and lysosomal complexes. Recent studies have uncovered that LRP1 is expressed by neural stem cells, acts as a critical regulator of oligodendrocyte progenitor cells behavior and early astroglial differentiation and involved in their differentiation, further indicating its participation in the Notch pathway, as Notch pathway is central in radial glia differentiation.

### SUMMARY OF INVENTION

The present disclosure provides down-regulation of LRP1 using genetic means or related inhibitors can facilitate Notch signaling inhibition, so as to reduce leukemia cell invasion, migration, anchorage-independent cell growth and tumorigenesis. Therefore, the disclosure reveals the essential role of LRP1 in Notch signaling dependent disease, and provides a novel strategy for treating Notch signaling dependent disease such as T-cell acute lymphoblastic leukemia (T-ALL); meanwhile, the invention provides novel medicines for treating Notch signaling dependent disease, and further points out a new direction for screening medicine and therapeutic target for the treating Notch signaling dependent disease.

In one aspect, the disclosure relates to a novel method of treating Notch signaling-dependent disease. In certain embodiments, the present disclosure provides a method for treating Notch signaling-dependent disease by using a LRP1 inhibitor, which may be a polypeptide antagonist specifically against LRP1, an RNA polynucleotide specific to LRP1, or a small molecule compound inhibitor specific to LRP1.

In one aspect, the invention provides a LRP1 specific inhibitor for use in treating Notch signaling-dependent disease. The LRP1 inhibitor is selected from a polypeptide antagonist specifically against LRP1, an RNA polynucleotide specific to LRP1, or a small molecule compound inhibitor specific to LRP1.

In one aspect, the invention provides use of a LRP1 specific inhibitor in preparation of medicine for treating Notch signaling-dependent disease. The LRP1 inhibitor is a polypeptide antagonist specifically against LRP1, an RNA polynucleotide specific to LRP1, or a small molecule compound inhibitor specific to LRP1

In certain embodiments, polypeptide antagonist is LRPAP1 or LRPAP1 derivative thereof that can bind to LRP1 on the cell surface and prevent ligands from its binding. Preferably, the polypeptide antagonist is selected from LRPAP1 comprising an amino sequence of SEQ ID NO: 1 or 2, an amino acid sequence with at least 70%, 80%, 85%, 90%, 95%, 99%, or more identity to SEQ ID NO:1 or 2, or an amino acid sequence with addition, deletion and/or substitution of one or more amino acids compared with SEQ ID NO:1 or 2. and the LRPAP1 can bind to LRP1 on the cell surface, preventing ligands from its binding.

In certain embodiments, LRPAP1 derivative is a polypeptide comprising: an amino acid sequence of SEQ ID NO:3; an amino acid sequence an amino acid sequence with at least about 70%, about 80%, about 85%, about 90%, about 95%, about 99%, or more identity to SEQ ID NO:3; or an amino acid sequence with addition, deletion and/or substitution of one or more amino acids compared with SEQ ID NO:3; and the LRPAP1 derivatives can bind to LRP1 on the cell surface, preventing ligands from its binding.

In certain embodiments, LRPAP1 derivative is a polypeptide comprising: an amino acid sequence of SEQ ID NO:4; an amino acid sequence an amino acid sequence with at least about 70%, about 80%, about 85%, about 90%, about 95%, about 99%, or more identity to SEQ ID NO:4; or an amino acid sequence with addition, deletion and/or substitution of one or more amino acids compared with SEQ ID NO:4; and the LRPAP1 derivatives can bind to LRP1 on the cell surface, preventing ligands from its binding.

In one embodiment, the LRPAP1 derivative is a polypeptide comprising SEQ ID NO:4 (RAPm6). In another embodiment, the LRPAP1 or LRPAP1 derivative is a polypeptide without or with a tag at the C-terminal of N-terminal of any sequence of SEQ ID NO: 1-4. The tag is selected from c-Myc, His, HA, GST, MBP, Flag, and Arg6. In one specific embodiment, LRPAP1 derivative is a polypeptide of SEQ ID NO: 4.

In one embodiment, the LRPAP1 or LRPAP1 derivative is a polypeptide modified by PEG.

In one embodiment, the polypeptide antagonist is an antibody against LRP1.

In one embodiment, the RNA polynucleotide is selected from siRNA, shRNA, guide RNA, and miRNA. The guide RNA is SEQ ID NO: 5 (TGGAGGACAAGATCTACCGC).

In one embodiment, the Notch signaling-dependent disease is selected from leukemia e.g. T-acute lymphoblastic leukemia or Chronic lymphocytic leukemia, myeloma e.g. Multiple myeloma, lymphoma e.g. Hodgkin lymphoma, Burkitt lymphoma, Diffuse large B-cell lymphoma, Mantle cell lymphoma, Splenic marginal zone lymphoma, Follicular lymphoma, breast cancer, liver cancer, lung cancer, and lung adenocarcinoma cells. The leukemia is T-acute lymphoblastic leukemia or Chronic lymphocytic leukemia. In one specific embodiment, the disease is any type of leukemia.

In one embodiment, the subject is non-human mammal or human.

In other aspect, the invention provides a method of screening medicines for treating Notch signaling-dependent disease using LRP1 as the target, the method comprising: observing the effect of candidate medicine on the expression or activity level of LRP1, if the candidate medicine can inhibit expression or activity level of LRP1, then it indicates that the candidate medicine is a potential medicine for treating Notch signaling-dependent disease. In one embodiment, the Notch signaling-dependent disease is selected from leukemia, The leukemia is selected from acute lymphoblastic leukemia (ALL), chronic lymphocytic leukemia (CLL).

### BRIEF DESCRIPTION OF DRAWINGS

FIG. 1. shows the expression of LRP1 gene in the control group and leukemia patients was evaluated by sequencing. The leukemia patients and healthy individuals are represented by boxes, respectively. Num (T), number of leukemia patients; num (N), number of healthy individuals.

FIG. 2 shows LRP1 directly interacts with DLL3 and promotes its membrane localization and stability. (A-H) LRP1β interacts with DLL3 in cells and in vitro. (A, B) HEK293T cell lysates were incubated with IgG control and antibodies recognizing DLL3 (A) or LRP1α or LRP1β (B). Five percent lysate was used as the input control. Blots with antibodies recognizing actin, DLL3, LRP1α or LRP1β are shown. (C-F) Mapping the binding regions between LRP1β and DLL3. (C, D) Schematics of LRP1β (C) and DLL3 (D) domain deletion mutants for domain mapping assays. (E, F) HEK293T cells were cotransfected with (E) Myc-tagged DLL3 and cSFB-tagged wild-type or mutant LRP1β or (F) Myc-tagged LRP1β and cSFB-tagged wild-type or mutant DLL3. The cell lysates were incubated with S beads. Five percent lysate was used as the input control. Blots with antibodies recognizing the FLAG and MYC epitope tags and actin are shown. (G) Colocalization assay of LRP1 and DLL3. HEK293T cells were transfected with cSFB-LRP1β and Myc-DLL3 and subjected to immunofluorescence with an anti-Myc antibody against DLL3 (red), an anti-Flag antibody against LRP1β (green) and DAPI (blue) and visualized by microscopy. Scale bars, 10 μm. Quantitation of immunofluorescence colocalization of LRP1β and DLL3, Pearson's R value was calculated with ImageJ software, and Pearson's R value >0.5 was considered as good colocalization. (H) In vitro GST pull-down assay of LRP1β and DLL3. In vitro purified GST-LRP1β and SUMO-DLL3 were coincubated with glutathione sepharose for 2 h. The pull-down assays were subjected to SDS-PAGE followed by Coomassie bright blue staining. (I-L) LRP1 is required for DLL3 membrane localization and stability. (I) Wild-type or LRP1-KO HEK293T cells were subjected to immunofluorescence with an anti-DLL3 antibody (green) and DAPI (blue) and visualized by microscopy. Scale bars, 10 μm. (J) Wild-type or LRP1-KO HEK293T cells were subjected to western blotting with antibodies recognizing DLL3, LRP1β, NOTCH1 and actin. (K) Knocking out LRP1 had no prominent effect on NOTCH1 localization. Wild-type or LRP1-KO HEK293T cells were subjected to immunofluorescence with an anti-NOTCH1 antibody (green) and DAPI (blue) and visualized by microscopy. Scale bars, 10 μm. (L) Wild-type or LRP1-KO HEK293T cells were homogenized and separated into cytosolic and membrane fractions. Lysates were subjected to western blotting with antibodies recognizing DLL3, LRP1β, CAV1 and actin. (M, N) Knocking out LRP1 impairs Notch signaling. (M) The mRNA levels of LRP1 and the Notch pathway target genes in wild-type and LRP1-KO HEK293T cells were determined by RT-qPCR. (N) Wild-type or LRP1-KO HEK293T cells were cotransfected with HES1 or HES5 luciferase constructs, respectively, and a Renilla luciferase construct. The relative luciferase activities of Notch target genes were determined using a dual-luciferase assay and normalized to that of Renilla luciferase. (A, B, E-N, n=3). The data are shown as the mean±SEM from three independent experiments. P values were calculated using two-tailed Student's t-tests (* P<0.05).

FIG. 3 shows LRP1 homologues function as important Notch pathway upstream regulators in C. elegans and Drosophila. (A-E) Overexpression of Notch signaling receptor LIN-12 reverses the molting defect induced by lrp-1 RNAi in C. elegans. (A) Experimental workflow for examining the effects of lrp-1 RNAi in WT and lin-12 overexpression lines. (B) Molting defects induced by lrp-1 RNAi in WT worms were visualized by microscopy. All phenotypes correlated percentages was collected and presented, respectively. (C) RNAi efficiencies for both lines. (D) Overexpression of lin-12 rescues the molting defect induced by lrp-1 RNAi. (E) The percentages of molting defect in worms exposed to lrp-1 RNAi. These are two different transgenic lines potentially expressing varied copy number of lin-12 showed as oe1 and oe2. F-N″ Fluorescence micrographs of wing discs are shown. (F-P′) Knockdown of LRP1 in the posterior region of wing discs reduces endogenous D1 expression. (H-J″ and N-P′) RNAi downregulation of dlg induced D1 upregulation (I′), invasion cell migration (I″), and MMP1 induction (O′) were all suppressed by reducing LRP1 activity (J′, J″ and P′). (K-M′) LRP1 knockdown in the posterior region of wing discs reduces endogenous Cut expression. (Q-R″′) Fluorescence micrographs of posterior adult midgut were shown. 8 days of LRP1 knockdown in the ISCs increased ISC proliferation (Cherry positive) and EE number (Pros positive). (S) Quantification of relative Pros+ cells in Q″ and R″. (T) Quantification of PH3+ mitotic cells per gut in Q″′ and R″′.

FIG. 4 shows that knocking out LRP1 attenuates Notch signaling-dependent leukemia invasion, migration, and tumorigenesis. (A, B) LRP1 is overexpressed in leukemia cell lines. (A) Western blots with antibodies recognizing LRP1β and actin in various leukemia cell lines. (B, C) The protein levels of DLL3 and NOTCH1 were determined in wild-type and LRP1-KO HSB2 (B) or K562 (C) cells by western blotting. (D) Knocking out LRP1 attenuates the expression of Notch target genes. The mRNA levels of the Notch target genes in wild-type and LRP1-KO HSB2 cells were determined by RT-qPCR. (E) Knocking out LRP1 does not affect leukemia cell proliferation. Growth curves for wild-type and LRP1-KO HSB2 or K562 cells are shown. (F-U) Knocking out LRP1 attenuates the invasion (F-I), migration (J-M), colony formation (N-Q) and tumorigenesis (R-U) of leukemia cells. (F, G) Invasion abilities of wild-type and LRP1-KO HSB2 (F) or K562 (G) cells were measured using a three-dimensional culture system with Matrigel. Scale bars, 100 μm. (H, I) The numbers (H) and the average diameters (I) of spheres were measured. (J, L) Migration abilities of wild-type and LRP1-KO HSB2 (J) or K562 (L) cells were measured using a transwell migration assay. Scale bars, 100 μm. (K, M) The numbers of cells that migrated into the lower chamber in J and L were counted. (N, P) Anchorage-independent tumorigenesis abilities of wild-type and LRP1-KO HSB2 (N) or K562 (P) cells were measured using a soft agar colony formation assay. (O, Q) The numbers of colonies in N, P, were counted. (R) Xenograft tumor growth studies were performed using wild-type or LRP1-KO HSB2 cells. Mice were euthanized after 4 weeks of injection. The tumors were excised, photographed, and weighed. (S, T) The volumes (S) and weights (T) of the tumors were measured. (U) The levels of DLL3 protein in tumors in (R) were detected by western blotting. Blots with antibodies recognizing DLL3 and actin are shown. (V) Knocking out LRP1 attenuates the expression of Notch target genes. The mRNA levels of the Notch target genes in tumors derived from wild-type and LRP1-KO HSB2 cells were determined by RT-qPCR. (A, C-Q, n=3; b, n=48; R-U, n=5). The data are shown as the mean±SEM from the indicated numbers of independent experiments. P values were calculated using two-tailed Student's t-tests (* P<0.05, ** P<0.01, NS, not significant).

FIG. 5 shows that comparison of WT and LRP1-KO cells from multiple perspectives. (A) The viability of WT and LRP1-KO HEK293T cells was measured using LDH release assay, Triton X-100 was used as a positive control. (B-D) The influence of LRP1 KO on cell apoptosis in HEK293T, HSB2 and K562 cells was evaluated via Caspase3 level detection (B) and Annexin V/PI staining (C-D). (E) The lipid raft was isolated from both WT and LRP1-KO HEK293T cells and evaluated via western blotting using FLOT1 as lipid raft marker. (F) The cell membrane integrity was detected by Trypan blue staining of WT and LRP1-KO cells. (G-I) The cholesterol uptake ability was measured in WT and LRP1-KO via incubation with NBD-cholesterol for 48 h and the fluorescence intensity was detected using microplate reader. (J) The levels of other lipoprotein receptors were detected by western blotting. Blots with antibodies recognizing LRP4, VLDLR, APOER2 and actin are shown. (A-J, n=3). The data are shown as the mean±SEM from three independent experiments. P values were calculated using two-tailed Student's t-tests (* P<0.05, NS, not significant).

FIG. 6 shows that Overexpression of NICD1 rescues LRP1-KO phenotypes in leukemia cells but not in MDA-MB-231 cells. (A, C, H) Anchorage-independent tumorigenesis abilities of LRP1-KO and LRP1-KO+NICD1 HSB2 (A), K562 (C) or MDA-MB-231 (H) cells were measured using a soft agar colony formation assay. (M) Anchorage-independent tumorigenesis abilities of MDA-MB-231 breast cancer cells treated with the vehicle or 0.1 mg/mL GST-RAPm6 for 36 h were measured using a soft agar colony formation assay. (B, D, I, N) The numbers of colonies in A, C, E, G were counted. (E, J) Xenograft tumor growth studies were performed using LRP1-KO HSB2 (E) or MDA-MB-231 (J) cells. Mice were euthanized after 4 weeks of injection. The tumors were excised, photographed, and weighed. (F, G, K, L) The volumes (F, K) and weights (G, L) of the tumors in E, J, were measured. (A-D, H, I, M, N, n=3; E-G, J-L, n=5). The data are shown as the mean±SEM from the indicated numbers of independent experiments. P values were calculated using two-tailed Student's t-tests (* P<0.05, ** P<0.01, NS, not significant).

FIG. 7 shows that LRP1 antagonist RAPm6 inhibits tumorigenesis in human leukemia cells, mouse xenografts and leukemia models. (A-C) RAPm6 interacts with LRP1 and inhibits Notch signaling. (A) GST and GST-RAPm6 were expressed and purified in E. coli and subjected to SDS-PAGE followed by Coomassie bright blue staining. (B) In vitro GST pull-down assay of LRP1α and RAPm6. HEK293T cell lysates were coincubated with GST or GST-RAPm6 and glutathione sepharose for 2 h. Five percent lysate was used as the input control. The pull-down assays were subjected to SDS-PAGE followed by western blotting with antibodies recognizing LRP1α and actin. (C) HSB2 cells were treated with the vehicle or RAPm6. The mRNA levels of LRP1 and Notch pathway target genes were determined by RT-qPCR. (D-Q) RAPm6 inhibits cell viability (D) and tumorigenesis in human leukemia cells (E-H), mouse xenografts (I-L) and leukemia (M-Q) models. (D) HSB2, K562 and DND41 leukemia cells were treated with 0.1 mg/mL GST-RAPm6 for 36 h. Cell viabilities were analyzed using a CCK-8 assay. (E, G) Anchorage-independent tumorigenesis abilities of wild-type and LRP1-KO HSB2 (E) or K562 (G) cells were measured using a soft agar colony formation assay. (F, H) The numbers of colonies in E, G, were counted. (I) Xenograft tumor growth studies were performed using HSB2 cells. Mice were treated with the vehicle or GST-RAPm6 and euthanized 4 weeks after injection. The tumors were excised, photographed, and weighed. (J, K) The volumes (J) and weights (K) of the tumors were measured. (L) The weights of the mice were measured. (M, N) Mouse leukemia models were established in NOD-SCID mice by injecting PBS or HSB2 cells intravenously via the tail vein. Mice were treated with the vehicle or GST-RAPm6. The percentages of CD5+ leukemia cells in peripheral blood were measured weekly by flow cytometry analysis. (O) The survival time of mice in m was recorded. (P) Spleens from mice in m were excised, and representative pictures of each group are shown. (Q) Representative pictures of HE-stained spleens from P. Scale bars, 50 μm. (A-H, n=3; I-L, n=5; M-Q, n=10). The data are shown as the mean±SEM from the indicated numbers of independent experiments. P values were calculated using two-tailed Student's t-tests (* P<0.05, ** P<0.01, NS, not significant).

### DETAILED DESCRIPTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The articles “a”, “an”, and “the” are used herein to refer to one or to more than one (i.e., to at least one) of the grammatical object of the article. By way of example, “a polypeptide” means one polypeptide or more than one polypeptide.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Term “LRP1” used herein refers to proteins encoded by low density lipoprotein receptor-related protein LRP1 gene in C. elegans, Drosophila, or mammals. LRP1 gene.

Term “inhibitor” used herein refers to materials capable of lowering, reducing or eliminating the amount, particular function, and particular property of a target object. Said target object can be a protein, polypeptide, nucleic acid and the like, while said inhibitor affects the amount, particular function, and particular property of the target object either directly or indirectly so as to result in the corresponding lowering, reducing or eliminating of the amount, particular function, and particular property of the target object. Said inhibitor can be a protein, polypeptide, nucleic acid, small molecule compound and the like.

For example, term “LRP1 inhibitor” used herein refers to materials capable of lowering, reducing or eliminating the expression, transcription, translation of LRP1 gene, and/or stability of LRP1 protein produced therefrom, binding ability to protein etc., which includes but is not limited to a polypeptide antagonist against LRP1, inhibitory nucleotides specific to LRP1, antibodies against LRP1 protein, small molecule compound inhibitors capable of inhibiting LRP1 activity, and/or materials capable of inhibiting the interaction between LRP1 protein and other membrane proteins, and the like.

The term “LRPAP1” refers to an antagonist of low-density lipoprotein receptor-related protein 1 (LRP1). LRPAP1 can bind to LRP1 on the cell surface, preventing ligands from binding. The term “binding” or “binds” as used herein refers to a non-random binding reaction between two molecules, such as for example between a ligand and a receptor. LRPAP1 can be polypeptide comprising an amino acid sequence of SEQ ID NO:1 or 2 or an amino acid sequence at least about 70%, about 80%, about 85%, about 90%, about 95%, about 99%, or more identity to SEQ ID NO:1 or 2, or an amino acid sequence with addition, deletion and/or substitution of one or more amino acids compared with SEQ ID NO:1 or 2.

The term “LRPAP1 derivatives” refers to a truncated LRPAP1 or its mutation and the polypeptide can bind to LRP1 on the cell surface, preventing ligands from binding. For example, LRPAP1 derivatives may be a truncated LRPAP1 shown as an amino acid sequence of SEQ ID NO:3 or an amino acid sequence an amino acid sequence with at least about 70%, about 80%, about 85%, about 90%, about 95%, about 99%, or more identity to SEQ ID NO:2, or an amino acid sequence with addition, deletion and/or substitution of one or more amino acids compared with SEQ ID NO:2. LRPAP1 derivatives can also bind to LRP1 on the cell surface, preventing ligands from binding. LRPAP1 derivative can be RAPm6 shown as an amino acid sequence of SEQ ID NO:4 or an amino acid sequence with at least about 70%, about 80%, about 85%, about 90%, about 95%, about 99%, or more identity to SEQ ID NO:4, or an amino acid sequence with addition, deletion and/or substitution of one or more amino acids compared with SEQ ID NO:4.

SEQ ID NO: 1
YSREKNQPKPSPKRESGEEFRMEKLNQLWEKAQRLHLPPVRLAELHADLKIQER
DELAWKKLKLDGLDEDGEKEARLIRNLNVILAKYGLDGKKDARQVTSNSLSGTQED
GLDDPRLEKLWHKAKTSGKFSGEELDKLWREFLHHKEKVHEYNVLLETLSRTEEIHE
NVISPSDLSDIKGSVLHSRHTELKEKLRSINQGLDRLRRVSHOGYSTEAEFEEPRVIDL
WDLAQSANLTDKELEAFREELKHFEAKIEKHNHYQKQLEIAHEKLRHAESVGDGER
VSRSREKHALLEGRTKELGYTVKKHLQDLSGRISRARHNEL
Full length of LRPAP1,
SEQ ID NO: 2
MAPRRVRSFLRGLPALLLLLLFLGPWPAASHGGKYSREKNQPKPSPKRESGEEF
RMEKLNQLWEKAQRLHLPPVRLAELHADLKIQERDELAWKKLKLDGLDEDGEKEA
RLIRNLNVILAKYGLDGKKDARQVTSNSLSGTQEDGLDDPRLEKLWHKAKTSGKFSG
EELDKLWREFLHHKEKVHEYNVLLETLSRTEEIHENVISPSDLSDIKGSVLHSRHTELK
EKLRSINQGLDRLRRVSHQGYSTEAEFEEPRVIDLWDLAQSANLTDKELEAFREELKH
FEAKIEKHNHYQKQLEIAHEKLRHAESVGDGERVSRSREKHALLEGRTKELGYTVKK
HLQDLSGRISRARHNEL
Truncated LRPAP1,
SEQ ID NO: 3
MYSREKNQPKPSPKRESGEEFRMEKLNQLWEKAQRLHLPPVRLAELHADLKIQE
RDELAWKKLKLDGLDEDGEKEARLIRNLNVILAKYGLDGKKDARQVTSNSLSGTQE
DGLDDPRLEKLWHKAKTSGKFSGEELDKLWREFLHHKEKVHEYNVLLETLSRTEEIH
ENVISPSDLSDIKGSVLHSRHTELKEKLRSINQGLDRLRRVSHOGYSTEAEFEEPRVID
LWDLAQSANLTDKELEAFREELKHFEAKIEKHNHYQKQLEIAHEKLRHAESVGDGER
VSRSREKHALLEGRTKELGYTVKKHLQDLSGRISRARHNEL
RAPm6,
SEQ ID NO: 4
MYSREKNQPKPSPKRESGEEFRMEKLNQLWEKAQRLHLPPVRLAELHADLKIQE
RDELAWKKLKLDGLDEDGEKEARLIRNLNVILAKYGLDGKKDARQVTSNSLSGTQE
DGLDDPRLEKLWHKAKTSGKFSGEELDKLWREFLHHKEKVHEYNVLLETLSRTEEIH
ENVISPSDLSDIKGSVLHSRHTELKEKLRSINQGLDRLRRVSHOGYSTEAEFEEPRVID
LWDLAQSANLTDKELEAFREELKHFEAKIEKFNFCQKQLEIAFEKLRHAESVGDGER
VSRSREKFALLEGRCKELGYTVKKHLQDLSGRISRARHNEL

Term “antibody” used herein refers to any immunoglobulin or complete molecule and fragments thereof which binds to a specific epitope. Said antibody includes but not limited to polyclonal antibodies, monoclonal antibodies, chimeric antibodies, humanized antibodies, single chain antibodies, and fragments and/or parts of intact antibodies, as long as such fragments or parts retain the antigen binding capacity of the parent antibody. In this disclosure, for example, “antibody against LRP1” refers to monoclonal antibodies, polyclonal antibodies, single chain antibodies and immunological activie fragments or parts thereof capable of specific binding to LRP1 protein, or functional variants or functional fragments thereof. In this disclosure, terms such as “LRP1 antibody”, “antibody against LRP1”, and “anti-LRP1 antibody” are used interchangeably.

In this disclosure, “functional variant” refers to the protein or polypeptide of the invention with one or more amino acid modification in its amino acid sequence. The modification can be a “conservative” modification (wherein the substituted amino acid has similar structure or chemical property) or a “non-conservative” modification; similar modification also include addition or deletion of amino acid or both. However, neither the modification of amino acid residue nor the addition or deletion of amino acid would substaintially change or damage the biological or immunological activity and function of the original amino acid sequence. In this disclosure, similarly, “functional fragment” refers to any part of the protein or polypeptide of the invention, which retains the substantially similar or identical biological or immunological activity and function of the protein or polypeptide of which it is a part (the parent protein or polypeptide).

Term “RNA polynucleotide specific to LRP1” used herein refers to nucleotide capable of binding to and/or inhibiting expression of LRP1 gene. Typical inhibitory nucleotide includes but not limited to antisense oligonucleotides, triple helix DNAs, RNA aptamers, ribozymes, small interfering RNA (siRNA), short hairpin RNA (shRNA) and microRNA. These nucleotide compounds bind to said specific genes with higher affinity than other nucleotide sequences, so as to inhibit expression of the specific genes.

Term “small molecule compound” used herein refers to organic compounds with molecular weight less than 3 k dalton which can be either natural or chemically synthesized. Term “derivative” used herein refers to compounds generated by modifying the parent organic compound through one or more chemical reactions, which have similar structures as the parent organic compound and similar effects in their functions. Term “analogue” used herein refers to compounds which were not generated by chemically modifying the parent organic compound but are similar to the parent organic compound in structure and have similar effects in their functions.

Term “disease” used herein refers to Notch signaling dependent disease e.g. Notch signaling activated cancers. The cancer can be but not limited the T-acute lymphoblastic leukemia (Activating mutations of NOTCH1 in human T cell acute lymphoblastic leukemia. Science. 2004; 306: 269-71. CUTLL1, a novel human T-cell lymphoma cell line with t (7;9) rearrangement, aberrant NOTCH1 activation and high sensitivity to gamma-secretase inhibitors. Leukemia. 2006; 20: 1279-87), Chronic lymphocytic leukemia (NOTCH1 mutations influence survival in chronic lymphocytic leukemia patients. BMC Cancer. 2013; 13: 274), Multiple myeloma (Inhibition of Notch signaling induces apoptosis of myeloma cells and enhances sensitivity to chemotherapy. Blood. 2008; 111: 2220-9), lymphoma e.g. Hodgkin lymphoma (Activated Notch1 signaling promotes tumor cell proliferation and survival in Hodgkin and anaplastic large cell lymphoma. Blood. 2002; 99: 3398-403.), Burkitt lymphoma (Notch is an essential upstream regulator of NF-kappaB and is relevant for survival of Hodgkin and Reed-Sternberg cells. Leukemia. 2012; 26: 806-13), Diffuse large B-cell lymphoma (Gain-of-function mutations and copy number increases of Notch2 in diffuse large B-cell lymphoma. Cancer Science. 2009; 100: 920-926.), Mantle cell lymphoma (Whole transcriptome sequencing reveals recurrent NOTCH1 mutations in mantle cell lymphoma. Blood. 2012; 119: 1963-1971), Splenic marginal zone lymphoma (The coding genome of splenic marginal zone lymphoma: activation of NOTCH2 and other pathways regulating marginal zone development. J Exp Med. 2012; 209: 1537-51.), Follicular lymphoma (Molecular detection of t (14;18) (q32; q21) in follicular lymphoma. Methods Mol Biol. 2013; Recurrent Mutations of NOTCH Genes in Follicular Lymphoma. Blood. 2013; 122: 4253), breast cancer (Notch1 is involved in migration and invasion of human breast cancer cells), liver cancer (Differentiation-inducing therapeutic effect of Notch inhibition in reversing malignant transformation of liver normal stem cells via MET. Oncotarget 9, 18885-18895 (2018).), lung cancer (Alterations of the Notch pathway in lung cancer. Proc. Natl Acad. Sci. USA 106, 22293-22298 (2009).), lung adenocarcinoma cells (Notch-1 stimulates survival of lung adenocarcinoma cells during hypoxia by activating the IGF-1R pathway. Oncogene 29, 2488-2498 (2010). Oxygen concentration determines the biological effects of NOTCH-1 signaling in adenocarcinoma of the lung. Cancer Res. 67, 7954-7959 (2007).).

Term “therapeutic target” used herein refers to various materials that can be used to treat a certain disease and the target of the material in animal or human bodies. Treatment effects on said disease are obtainable when said materials act on said target. Said materials can be a variety of materials such as protein, polypeptide, nucleic acid, small molecule compound, said target can be material substances such as a certain gene (including a specific sequence of a gene), a certain protein (including a specific site of a protein), a certain protein complex (including specific binding site thereof), or certain charactistics, certain functions, certain interaction relationships with peripheral substances and environment of aforementioned genes and/or proteins, etc, as long as said materials can affect the gene, protein, protein complex, or charactistic, function, interaction relationship thereof so as to treat the disease.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

Terms “treat”, “treating”, or “treatment” used herein refer to reversing, ameliorating or inhibiting the progression of the disease to which the term is applied, or one or more symptoms of the disease. As used herein, depending on the condition of the patient, the term also include prevention of disease, which includes the prevention of disease or the onset of any symptoms associated therewith, and ameliorating symptoms or reducing the severity of any condition before its onset.

Terms “inhibit”, “weaken”, “down-regulate”, “remove” and the like all refer to reduction or decreasing in quantity or degree. Such reduction or decreasing is not limited to any extent as long as it exhibits such a trend. For example, the reduction or decreasing can be 100% relative to the original quantity or degree, or can be 50% or even 1% or less.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215: 403-410 (1990); Stephen F. et al., Nucleic Acids Res., 25: 3389-3402 (1997)), ClustalW2 (available on the website of European Bioinformatics Institute, see also, Higgins D. G. et al., Methods in Enzymology, 266: 383-402 (1996); Larkin M. A. et al., Bioinformatics (Oxford, England), 23 (21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

The present disclosure will be further illustrated in detail below. However, ways to carry out the present invention are not limited to the following examples.

KEY RESOURCES TABLE

| | | |
|---|---|---|
| REAGENT or RESOURCE | SOURCE | IDENTIFIER |

Antibodies

| | | |
|---|---|---|
| LRP1α | Abcam | ab20384 |
| LRP1B | Abcam | ab92544 |
| DLL3 | Abcam | ab103102 |
| Actin | Genescript | A00702 |
| FLAG | Sigma | B3111 |
| Myc | Genescript | A00704 |
| Cleaved-NOTCH1 | Cell Signaling Technology | #3608 |
| CAV1 | Cell Signaling Technology | #3858 |
| HA | Genescript | A01244 |
| Goat Anti-Mouse IgG Antibody | Genescript | A00160 |
| (H&L) [HRP] | | |
| Goat Anti-Rabbit IgG Antibody | Genescript | A00178 |
| (H&L) [HRP] | | |
| NOTCH1 | Bethyl | A301-895A |
| EEA1 | Cell Signaling Technology | #3288 |
| GOPC | Cell Signaling Technology | #8576 |
| STX6 | Cell Signaling Technology | #2869 |
| RAB11 | ABclonal | A3251 |
| ApoER2 | HuaBio | ER1803-10 |
| VLDLR | HuaBio | ER1918-54 |
| DLL4 | HuaBio | ER1706-29 |
| DLL1 | ABclonal | A14277 |
| Caspase-3 | HuaBio | ER1802-42 |
| LRP4 | Immunoway | YN0833 |
| FLOT1 | Abcam | ab133497 |
| GAPDH | Abcam | ab8245 |
| DAPI | Cell Signaling Technology | #4083 |
| Goat Anti-Rabbit IgG H&L | Abcam | ab150077 |
| (Alexa Fluor ® 488) | | |
| Goat Anti-Mouse IgG H&L | Abcam | ab150116 |
| (Alexa Fluor ® 594) | | |
| FluorSave ™ Reagent | Millipore | #345789 |
| D1 | Developmental Studies Hybridoma | C594.9B |
| | Bank | |
| MMP1 | Developmental Studies Hybridoma | 3A6B4: |
| | Bank | 3B8D12: |
| | | 5H7B11=1:1:1 |
| | | mixed |
| Pros | Developmental Studies Hybridoma | MR1A |
| | Bank | |
| PH3 | Cell Signaling Technology | #9701 |
| rabbit Alexa | Cell Signaling Technology | #4412 |
| mouse Alexa | Cell Signaling Technology | #4408 |
| mouse Cy3 | Jackson ImmunoResearch | #715-165-150 |
| FITC mouse anti-human CD5 | BD Pharmingen | #561896 |

Chemicals

| | | |
|---|---|---|
| FBS | Gibco | C11875500CP |
| Penicillin and streptomycin | Thermo Fisher Scientific | #15140163 |
| Membrane/soluble protein | Beyotime | P0033-1 |
| isolation kit | | |
| TurboNuclease | Accelagen | N0103M |
| Streptavidin-conjugated beads | GE | #17-5113-01 |
| Biotin | Sigma | B4501 |
| Trizol | Thermo Fisher Scientific | #15596026 |
| Highscript III reverse | Vazyme | R212-02 |
| transcriptase | | |
| TB SYBR Green Master Mix | Takara | RR820 |
| S-protein beads | Millipore | #69704 |
| Matrigel | Corning | #356237 |
| RBC lysis | Solarbio | R1010-100m1 |
| Cholesterol Uptake Assay Kit | Abcam | ab236212 |
| Trypan Blue solution | Thermo Fisher Scientific | T10282 |
| LDH Cytotoxicity Assay Kit | Beyotime | C0016 |
| Lipid Raft Isolation Kit | Invent Biotech | LR-039 |
| Annexin V-FITC Apoptosis | Beyotime | C1062M |
| Detection Kit | | |
| Dual-Glo Luciferase assay | Promega | E2920 |
| system | | |
| Sulfo-NHS-SS-Biotin | Apexbio | A8005-100 |

Vector

| | | |
|---|---|---|
| pDONR201 | Invitrogen | N/A |
| pDEST-cSFB | This paper | N/A |
| pDEST-Myc | This paper | N/A |
| pLenti-V2 | Addgene | #52961 |

C. elegans related source

| | | |
|---|---|---|
| N2 Bristol | This paper | N/A |
| WU45 Ex | This paper | N/A |
| [glp-1p::lin-12::mRFP3-myc | | |
| myo-2p::GFP] | | |
| E. coli HT115 | Caenorhabditis Genetics Center | N/A |
| L4440 | This paper | N/A |
| pPD95.77 | This paper | N/A |
| lrp-1 RNAi #1 | Ahringer library | lrp-1 |
| | | 1-2000 bp |
| lrp-1 RNAi #2 | Ahringer library | lrp-1 |
| | | 2000-3000 bp |

Drosophila related source

| | | |
|---|---|---|
| ptc-GAL4 | Bloomington Drosophila Stock | N/A |
| | Center | |
| en-GAL4 | Bloomington Drosophila Stock | N/A |
| | Center | |
| hh-GAL4 | Bloomington Drosophila Stock | N/A |
| | Center | |
| LRP1^{EY07878} (16864) | Bloomington Drosophila Stock | N/A |
| | Center | |
| UAS-GFP | Bloomington Drosophila Stock | N/A |
| | Center | |
| UAS-LRP1.RNAi (#1, v8397) | Vienna Drosophila Resource | N/A |
| | Center | |
| UAS-dlg.RNAi (v41136) | Vienna Drosophila Resource | N/A |
| | Center | |
| UAS-LRP1.RNAi | Tsinghua Fly Center, Tsinghua | N/A |
| (#2, THU3999) | University | |
| esg-GAL4 | Vienna Drosophila Resource | N/A |
| | Center | |
| UAS-mCherry | Vienna Drosophila Resource | N/A |
| | Center | |
| tub-Gal80^{ts} | Vienna Drosophila Resource | N/A |
| | Center | |
| Su(H)Gbe-GAL80 | A gift from Hangsong Deng, | N/A |
| | Tongji University, Shanghai, | |
| | China | |

Primer

| | |
|---|---|
| qPCR-LRP1-F | 5-CTATCGACGCCCCTAAGACTT |
| qPCR-LRP1-R | 5-CATCGCTGGGCCTTACTCT |
| qPCR-HES2-F | 5-CCAACTGCTCGAAGCTAGAGA |
| qPCR-HES2-R | 5-AGCGCACGGTCATTTCCAG |
| qPCR-HES5-F | 5-CGCATCAACAGCAGCATCGAG |
| qPCR-HES5-R | 5-GACGAAGGCTTTGCTGTGCT |
| qPCR-c-MYC-F | 5-GGCTCCTGGCAAAAGGTCA |
| qPCR-c-MYC-R | 5-CTGCGTAGTTGTGCTGATGT |
| qPCR-Actin-F | 5-TTGCCGACAGGATGCAGAAGGA |
| qPCR-Actin-R | 5-AGGTGGACAGCGAGGCCAGGAT |
| sg-LRP1 | 5-TGGAGGACAAGATCTACCGC |
| Drosophila lrp1-F | 5-ATCAGGTGCCAGAGTCGTC |
| Drosophila lrp1-R | 5-CCCGCGAAAGAAAAATCAGCG |
| Drosophila rp49-F | 5-TCCTACCAGCTTCAAGATGACC |
| Drosophila rp49-R | 5-CACGTTGTGCACCAGGAACT |
| C. elegans actin-F | 5-TGCTGATCGTATGCAGAAGG |
| C. elegans actin-R | 5-TAGATCCTCCGATCCAGACG |
| C. elegans actin-F | 5-CTGGACAATCTGAATGCCTTG CAATG |
| C. elegans actin-R | 5-CCATTTACTAGTTTTTGACCAATT GGAC |

### EXPERIMENTAL MODEL AND SUBJECT DETAILS

### Cell Culture, Plasmid Construction, Transfection and Lentivirus Packaging

HEK293T and MDA-MB-231 cells were cultured in Dulbecco's modified Eagle's medium (DMEM, Gibco, Australia) supplemented with 10% fetal bovine serum. JURKAT, K562, DND-41, ICHIKAWA and HSB2 cells were cultured in RPMI 1640 medium supplemented with 10% fetal bovine serum. All culture media contained 10% fetal bovine serum (FBS, Gibco, Australia) and were supplemented with 1% penicillin and streptomycin (Thermo Fisher Scientific, USA).

cDNAs encoding all of the genes were obtained from the hORFV5.1 library or amplified from cDNA of HEK293T cells using RT-PCR. cDNAs were subcloned into the pDONR201 vector (Invitrogen, USA) as entry clones and subsequently transferred to Gateway-compatible destination vectors for the expression of C-terminal streptavidin-binding peptide (SFB) triple tagged- (S protein tag-2×FLAG tag-SBP tag) or MYC-tagged fusion proteins. Deletion mutants of LRP1 and DLL3 were generated using site-directed mutagenesis. sgRNA against LRP1 was synthesized and cloned into the pLenti-V2 vector (Addgene #52961). All constructs were confirmed by sequencing.

Constructs encoding C-terminal SFB-tagged proteins were transfected into HEK293T cells using polyethyleneimines. For knocking out experiments, sgRNA constructs were packaged into lentiviruses by co-transfecting them with the packaging plasmids pMD2G and pSPAX2 into HEK293T cells. Forty-eight hours after transfection, the supernatant was collected and used to infect HEK293T, MDA-MB-231, HSB2 or K562 cells. Infections were repeated twice at an interval of 24 h to achieve maximal infection efficiency. Stable cells were selected using medium containing 2-5 μg/mL puromycin. Overexpression or knocking out efficiencies were confirmed using Western blot analysis.

### TAP of SFB-Tagged Protein Complexes

We picked 12 clones of each bait for the follow-up experiments and confirmed the bait protein expression and localization using Western blotting and immunostaining, respectively. From which, we chose two clones of each bait with the correct subcellular localization and a moderate expression close to that of endogenous one as biological repeat for TAP-MS analysis. HEK293T cells stably expressing C-terminal SFB-fused Notch pathway proteins were selected via culture in medium containing 2 μg/ml puromycin. Protein expression was confirmed via immunostaining and Western blotting as described previously (Wang, W., Li, X., Huang, J., Feng, L., Dolinta, K. G., and Chen, J. (2014). Defining the protein-protein interaction network of the human hippo pathway. Mol Cell Proteomics 13, 119-131.).

For TAP experiments, the membrane-bound and soluble proteins of 2×10⁸ HEK293T cells were extracted using a membrane/soluble protein isolation kit (Beyotime, China) with protease inhibitors at 4° C. The insoluble pellets from the crude lysis step were briefly sonicated and incubated with TurboNuclease for 30 min at 37° C. with occasional vortexing to extract chromatin-bound protein complexes. The lysates were then centrifuged at 14,000 rpm for 30 min at 4° C., and the supernatant was collected as a chromatin fraction. All three fractions were combined and incubated with streptavidin-conjugated beads (GE, USA) for 2 h at 4° C. The beads were washed three times with NETN buffer, and bound proteins were eluted with NETN buffer containing 2 mg/mL biotin (Sigma, USA) for 2 h at 4° C. The elutes were incubated with S-protein beads (EMD Millipore, USA) for 1 h. The beads were washed three times with NETN buffer and subjected to sodium dodecyl sulphate-polyacrylamide gel electrophoresis (SDS-PAGE). Each pull-down sample was run just in the separation gel so that the whole band could be excised as one sample and subjected to in-gel trypsin digestion and LC-MS.

### LC and MS

LC and MS were performed as described previously (Li, X., Han, H., Zhou, M. T., Yang, B., Ta, A. P., Li, N., Chen, J., and Wang, W. (2017). Proteomic Analysis of the Human Tankyrase Protein Interaction Network Reveals Its Role in Pexophagy. Cell Rep 20, 737-749.; 31. Li, X., Tran, K. M., Aziz, K. E., Sorokin, A. V., Chen, J., and Wang, W. (2016). Defining the Protein-Protein Interaction Network of the Human Protein Tyrosine Phosphatase Family. Mol Cell Proteomics 15, 3030-3044.). Briefly, the excised gel bands described above were cut into approximately 1-mm³ pieces, which were then subjected to in-gel trypsin digestion (Shevchenko, A., Wilm, M., Vorm, O., and Mann, M. (1996). Mass spectrometric sequencing of proteins silver-stained polyacrylamide gels. Anal Chem 68, 850-858.) and dried. Samples were reconstituted in 5 μL of high-performance liquid chromatography solvent A (2.5% acetonitrile and 0.1% formic acid). A nanoscale reverse-phase high-performance liquid chromatography capillary column was created by packing 5-μm C18 spherical silica beads into a fused silica capillary (100 μm inner diameter×˜20 cm length) using a flame-drawn tip. After the column was equilibrated, each sample was loaded onto the column using an autosampler. A gradient was formed, and peptides were eluted with increasing concentrations of solvent B (97.5% acetonitrile and 0.1% formic acid).

As the peptides eluted, they were subjected to electrospray ionization and then analyzed by an Orbitrap Fusion Lumos Tribrid Mass Spectrometer (Thermo Fisher Scientific, USA). The source was operated at 1.9 kV, with no sheath gas flow and with the ion transfer tube at 350° C. The data-dependent acquisition mode was used. The survey scan was conducted from m/z 350 to 1,500, with a resolution of 60,000 at m/z 200. The 20 most intense peaks with charge states of 2 and greater were acquired with collision-induced dissociation with a normalized collision energy of 30% and one micro scan; the intensity threshold was set at 1,000. MS2 spectra were acquired with a resolution of 15,000. The peptides were detected, isolated, and fragmented to produce a tandem mass spectrum of specific fragment ions for each peptide. Peptide sequences and, hence, protein identity was determined by matching fragmentation patterns in protein databases using the Mascot software program (Matrix Science, USA). Enzyme specificity was set to partially tryptic with two missed cleavages. Modifications of the peptides included carboxyamidomethyl (cysteines, variable) and oxidation (methionine, variable). Mass tolerance was set to 20 ppm for both precursor ions and fragment ions. The database searched was Swiss-Prot (Homo sapiens). Spectral matches were filtered to contain the false-discovery rate to less than 1% at the peptide level using the target-decoy method (Elias and Gygi, 2007), and protein inference was considered following the general rules (Nesvizhskii and Aebersold, 2005) with manual annotation applied when necessary. This same principle was used for protein isoforms when they were present. Generally, the longest isoform was reported.

### MS Data Analysis and Bioinformatic Analysis

MS data analysis was performed using the MUSE algorithm as described previously to assign quality scores for the identified PPIs. Twenty-two unrelated TAP-MS experiments using overexpressed TAP-tagged protein baits performed under identical experimental conditions were used as controls for the MUSE analysis. A MUSE score was assigned to each identified interaction, and any interaction with a MUSE score of at least 0.85 and raw spectral counts greater than 1 was considered to be an HCIP. To estimate the potential false-positive rate, our HCIP data set was compared with the frequency and abundance information in the CRAPome database (V1.1, 2014.1) (Mellacheruvu, D., Wright, Z., Couzens, A. L., Lambert, J. P., St-Denis, N. A., Li, T., Miteva, Y. V., Hauri, S., Sardiu, M. E., Low, T. Y., et al. (2013) The CRAPome: a contaminant repository for affinity purification-mass spectrometry data. Nat Methods 10, 730-736.). We also searched the binary interactions involving HCIPs in eight knowledge databases, including BioGRID, STRING, IntAct, MINT, HI-union, HuRI, Lit-BM and HI-II-14, to find the literature-reported interactions in the HCIPs.

The overall and individual interactomes of Notch pathway core components were enriched in signaling pathway and functional categories using the HCIP data sets. P values were estimated using the Knowledge Base included with the Ingenuity Pathway Analysis software program (Ingenuity Systems, USA), which contained findings and annotations from multiple sources, including the Gene Ontology, KEGG pathway, and PANTHER Pathway databases. Only statistically significant correlations (P<0.05) are shown. The −log (P value) for each function and related HCIPs is listed. CHD patient data sets were downloaded from previous study (Jin, S. C., Homsy, J., Zaidi, S., Lu, Q., Morton, S., DePalma, S. R., Zeng, X., Qi, H., Chang, W., Sierant, M. C., et al. (2017). Contribution of rare inherited and de novo variants in 2,871 congenital heart disease probands. Nat Genet 49, 1593-1601.). Cancer patient data sets were downloaded from cBioPortal and an exome sequencing data of 130 T-ALL patients.

### Western Blot, Pull-Down and Co-Immunoprecipitation

Whole-cell lysates were prepared by lysing cells with NETN buffer (20 mM Tris-HCl, pH 8.0, 100 mM NaCl, 1 mM EDTA, 0.5% Nonidet P-40) on ice for 30 min and then boiling them in 2× Laemmli buffer. To extract chromatin-bound protein complexes while minimizing the interactions mediated by DNA, we treated insoluble pellets resulting from crude lysis with TurboNuclease (Accelagen), which hydrolyses single- and double-stranded DNA and RNA to oligonucleotides that are 1-4 bases long to release chromatin-bound proteins (i.e., the chromatin fraction). This step disrupts any protein-protein interactions that may be mediated by DNA. The same protocol was used to prepare lysates for our co-IP experiments using chromatin fractions. Lysates were subjected to SDS-PAGE followed by immunoblotting with antibodies against various proteins as indicated.

For in cell pull-down and co-IP assays, 1×10⁷ cells were lysed with NETN buffer on ice for 30 min. The lysates were then incubated with 20 μl of conjugated beads (for SFB-tagged pull-down) for 2 h at 4° C. or incubated with antibodies against endogenous proteins for 1 h at 4° C. followed by the addition of 20 μL of protein A/G agarose and incubation for 2 h at 4° C. Beads were washed three times with NETN buffer and boiled in 2× Laemmli buffer. Lysates were subjected to SDS-PAGE followed by WB. For in vitro pull-down assays, GST or GST-LRP1β were first incubated with GST resin for 2 h in 4° C., then purified SUMO-DLL3 protein was added after phosphate-buffered saline (PBS) wash three times and incubated for additional 2 h in 4° C. Beads were washed three times with PBS and boiled in SDS loading buffer. Lysates were subjected to SDS-PAGE followed by Coomassie bright blue stain or WB.

### Immunofluorescence

For immunofluorescence assays, cells were seeded in a cell culture dish, fixed with 4% paraformaldehyde at room temperature for 10 min. Cells were permeabilized 10 min with 0.1% TritonX-100, washed with PBS and blocked in 5% BSA in PBS for 30 minutes before labelling in primary antibodies at room temperature for 1 h. and permeabilized at 4° C. for 30 min. After incubation with the indicated antibodies at room temperature for 1 h, the cells were washed with PBS twice, stained with goat-anti-rabbit Fluorescein isothiocyanate-labelled IgG or goat-anti-mouse rhodamine-labelled IgG (1:5000, Abcam, UK) at room temperature for 1 h, and subjected to 4′,6-diamidino-2-phenylindole (DAPI) staining (Sigma-Aldrich, USA). Coverslips were mounted using FluorSave™ Reagent (Milipore, USA). The cells were viewed using an Olympus IX73 Microscope Imaging System (Olympus, Japan).

Quantitative Real-Time PCR (qPCR)

Cells were harvested, and total RNA was extracted using a Trizol reagent (Thermo Fisher Scientific, USA). RNA from each sample was reverse-transcribed into cDNA using Highscript III reverse transcriptase (Vazyme, China). Levels of mRNA for specific genes were quantified by qPCR using a Qtower3G qPCR system (Jena Bioscience, Germany) with SYBR Green Master Mix (Takara, Japan). The data were normalized to the Actin expression level in each sample.

Nematode Strains and Feeding RNAi in C. elegans

Two nematode strains were grown at 20° C., and maintained following standard procedures (Brenner, 1974). N2 Bristol was used as the wild-type strain. Another strain is: WU45 Ex [glp-1p::lin-12::mRFP3-myc myo-2p::GFP].

E. coli HT115 was obtained from the Caenorhabditis Genetics Center, L4440 as empty vector. The lrp-1 RNAi clone from Ahringer library was used in this study and fed to C. elegans as described previously (Wu, L., Zhou, B., Oshiro-Rapley, N., Li, M., Paulo, J. A., Webster, C. M., Mou, F., Kacergis, M. C., Talkowski, M. E., Carr, C. E., et al. (2016). An Ancient, Unified Mechanism for Metformin Growth Inhibition in C. elegans and Cancer. Cell 167, 1705-1718 e1713.). The P0 animals of N2 and WU45 were grown on L4440 bacteria from L4 stage. When reaching D2 adult stage, gravid animals were used for egg laying for 2 hours on L4440 and lrp-1 RNAi respectively. After 72 hours of RNAi feeding, the number of animals with molting defect were counted, analyzed relative to total worms, and imaged using a Leica DM500 microscope at a magnification of 10×.

### Construction of the Lin-12 Transgenic Line and Microinjection

For the transgenic line, the genomic sequence of lin-12 gene and 990 bp of the glp-1 promoter sequence were cloned into a plasmid vector pPD95.77 with monomeric red fluorescent protein (mRFP) following a C-terminal Myc tag. Plasmid (20 ng/μL) and injection marker myo-2p::GFP (3 ng/μl) were injected into the gonad of wild type adult animals and screened for the transgenic line according to fluorescence.

### Drosophila Stocks and Immunostaining

All crosses were raised on standard Drosophila medium. The following strains from the Bloomington Drosophila Stock Center were used for this study: ptc-GAL4, en-GAL4, UAS-GFP, hh-GAL4, LRP1EY07878 (16864). The following RNAi lines were collected from the Vienna Drosophila Resource Center: UAS-LRP1.RNAi (v8397), UAS-dlg.RNAi (v41136). UAS-LRP1.RNAi (#2, THU3999) was obtained from Tsinghua Fly Center, Tsinghua University, Beijing China. esg-GAL4, UAS-mCherry, tub-Gal80ts; Su(H)Gbe-GAL80 was a gift from Hangsong Deng, Tongji University, Shanghai, China.

Fluorescently labeled clones were produced in the eye discs by crossing FRT42D or FRT42D, LRP1EY07878 with the following strain: FRT42D, tub-Gal80; ey-Flp6, Act>y+>Gal4, UAS-GFP (42D tester). Wing and eye imaginal discs of third-instar larvae were dissected in PBS and fixed in PBS containing 4% formaldehyde for 15 min, and fly intestines were fixed for 40 min. The samples were then blocked in 1×PBS-Tween 20 containing 5% normal goat serum for 1 h and were incubated first with primary antibody overnight at 4° C. with the following primary antibodies: mouse anti-MMP1 (1:100), mouse anti-D1 (1:100), mouse anti-Cut (1:100), mouse anti-Pros [1:100, Developmental Studies Hybridoma Bank (DSHB)], rabbit anti-PH3 [1:200; cell signaling technology (CST)]. Samples were then incubated with fluorescence-conjugated secondary antibodies for 2 h at room temperature. Flies used for gut dissection were reared at 18° C. and 3-day-old adult female of the indicated genotypes were shifted to 29° C. to inactivate temperature sensitive GAL80 (GAL80ts) and allow expression of the transgenes for 8 or 14 days. For D1 and MMP1, and Cut staining, larvae were shift to 29° C. one day after egg laying.

### Quantitative Real-Time PCR Analysis of Drosophila Adult Heads

Drosophila adult heads of indicated genotypes using two independent LRP1 RNAi strains were removed and total RNA was then extracted using TRIzol (Ambion). Total RNA was reverse-transcribed into cDNA with the HiScript II 1st Strand cDNA Synthesis Kit (Vazyme); and quantitative PCR was performed with KAPA SYBR® FAST (KAPA BIOSYSTEMS) and quantified by the QuantStudio™ 5 Real-Time PCR System (ThermoFisher). RP49 was used as an internal control.

### Cholesterol Uptake

The cholesterol uptake ability was evaluated using Cholesterol Uptake Assay Kit (ab236212, Abcam) according to the manufactures guidelines. Briefly, cells were seeded at a density of 5×10⁵ cells/mL and incubated overnight in serum-free medium with 20 μg/mL NBD Cholesterol in a cell culture incubator at 37° C. The next day, culture medium was removed and replaced with an appropriate volume of assay buffer, then the degree of NBD cholesterol uptake was analyzed using a microplate reader.

### Trypan Blue Staining

Trypan blue staining was used to determine the cell viability as follows. First, the cell suspension was prepared and then incubated with 0.4% Trypan Blue solution (T10282, Thermo Fisher Scientific) at 1:1 ratio for 1-2 minutes at room temperature. Non-viable cells will be blue, viable cells will be unstained. Cell staining was observed under a light microscope and positively stained cell was calculated.

### LDH Release

Lactate dehydrogenase (LDH) assay. To evaluate the integrity of cell membrane, the LDH release from these cell lines was measured using LDH Cytotoxicity Assay Kit (Beyotime) based on the manufacturer's instruction. Briefly, both wild-type and LRP1-KO cell lines were collected and washed once with fresh regular culture medium, then seeded into a 96-well plate with 2-10×10⁴ cells/well. After Incubating in an incubator (5% CO2, 90% humidity, 37° C.) for the appropriate time of treatment, cells were centrifuged at 400×g for 5 min to precipitate and the clear medium solution (120 μL/well) was transferred into an optically clear 96-well plate for detection.

### Isolation of Lipid Raft

Membrane lipid raft was isolated using Minute™ Total Lipid Raft Isolation Kit (Invent Biotech) according to manufactures' guidelines. 30-40×10⁶ cells were collected by low speed centrifugation (500-600× g for 5 min) and washed once with cold PBS. Remove supernatant completely and resuspend the pellet in buffer A, incubating on ice for 5 min. Vortex the tube vigorously for 10-30 seconds. Immediately transfer the cell suspension to the filter cartridge. After a series of centrifugation, pellet containing total membrane fraction was acquired and resuspended in buffer B and C consecutively. Finally, the lipid rafts would adhere to the wall of the microfuge tube after removal of the aqueous phase and resuspended in 50-200 μL buffer for following western blotting analysis.

### Annexin V/PI Staining

Cell apoptosis was evaluated using Annexin V-FITC Apoptosis Detection Kit (Beyotime) according to the manufactures' instructions. Briefly, 1-5×10⁵ cells were collected by centrifugation, followed by washing with cold 1× PBS and carefully remove the supernatant. Resuspend the cells in 195 μL Annexin V-FITC binding buffer and add 5 μL of Annexin V-FITC and 10 μL propidium iodide (PI) staining solution to tubes and gently swirl to mix. After incubating the mixture for 20 minutes at room temperature in the dark, cells were immediately analyzed by flowcytometry.

### Luciferase Reporter Assay

The HES1 and HES5 promoter driven-luciferase reporter constructs were generated by insertion of the HES1 and HES5 promoter into pGL3-luc luciferase vector upstream of the firefly luciferase gene. For luciferase assay, cells were plated at 50% confluency in 24-well plate and grown overnight. The firefly luciferase reporter construct and the Renilla control reporter were contransfected into the cells at a molar ratio of 10:1. After 24 h of culture, the luciferase reporter activity was assayed with the Dual Luciferase Assay System (Promega).

For coculture system, WT and DLL3 KD cells were plated in a six-well plate overnight and then cotransfected with the luciferase reporter and Renilla control reporter as an internal control. Twenty-four hours after transfection, the transfected cells were cocultured with DLL1 overexpressed cells for additional 24 hr. In another case, WT cells were first cotransfected with the luciferase reporter and Renilla control reporter and then cocultured with WT and DLL3 KD cells respectively. Luciferase activities were measured using a Dual Luciferase Assay System (Promega).

### Cell Surface Biotinylation

Biotinylation-streptavidin pull down was performed essentially as described previously. Briefly, 293T cells expressing Myc-DLL3 were labeled on 4° C. for 30 min with Sulfo-NHS-SS-Biotin solution (0.5 mg/mL in PBS). After a 30 min incubation at 4° C. or 37° C., biotin was stripped by twice 30 min incubation with 10 mM DTT in TNEB buffer (20 mM Tris, pH 8.3; 150 mM NaCl; 1 mM EDTA; 0.2% BSA) on ice. Cells were lysed with RIPA, biotinylated species were purified on streptavidin agarose and analyzed by immunoblotting with anti-Myc antibody

### Cell Invasion, Migration and Colony Formation Assays

Leukemia cells invasion was measured using a three-dimensional culture system with Matrigel (Corning, USA). 5,000 wild-type or LRP1-KO HSB2 or K562 cells were mixed with 500 μL Matrigel and plated in 24-well plates. The spheres were visualized by microscopy 7 days post seeding. The numbers and the average diameters of spheres were measured using a GelDoc with Quantity One software (Bio-Rad, USA).

Leukemia cells migration was measured using a transwell migration assay. 50,000 wild-type or LRP1-KO HSB2 or K562 cells were seeded onto a 24-well transwell chamber. Cells migrated into the lower chamber were visualized by microscopy 36 h post seeding. The numbers of cells migrated into the lower chamber were counted manually.

For soft agar colony formation assays, 3,000 wild-type or LRP1-KO HSB2 or K562 cells were added to 1.5 mL of growth medium with 1.4% agar and layered onto 2 mL of 2.4% agar bed in 6-well plates. Medium was replenished every week for 4 weeks. Resulting colonies were fixed and stained with 0.005% crystal violet solution overnight and photographed. The numbers of colonies were counted with a GelDoc with Quantity One software (Bio-Rad, USA).

### Mouse Xenograft and Leukemia Models

All animal experiments were performed in accordance with a protocol approved by the Institutional Animal Care and Use Committee of the Westlake University. For mouse xenograft models, 5×10⁶ each types of the cells (eg. HSB2 vs HSB2 LRP1-KO) were resuspended in 100 μL of Matrigel diluted with PBS at 1:1 ratio and injected subcutaneously into left and right flanks of 5 anesthetized 6-week-old female BALB/c nude mice respectively. Starting from the day 7, the tumor formation was observed weekly and tumor sizes were measured. Mice were euthanized after 4 weeks of injection and the tumors were excised, photographed and weighed.

Mouse leukemia models were established in NOD-SCID mice. 5×106 HSB2 cells were resuspended in 100 μL PBS and injected intravenously into 6-week-old female NOD-SCID mice via tail vein. Starting from the day 7, vehicle or RAPm6 protein was administered by tail vein injection for 3 days followed by 4 days of rest for a total of 4 cycles. After each cycle, peripheral blood leukemia cells were analyzed using flow cytometry as follows. Peripheral blood was collected from treated NOD-SCID mice and red blood cells were removed using RBC lysis (Beyotime, China). After washing three times with PBS, cells were labelled in suspension with FITC mouse anti-human CD5 (BD Pharmingen, USA) for 30 min at 4° C. Cells were then washed three times with PBS and analyzed on an CytoFLEX6 flow cytometer with CytExpert software as recommended to the manufacturer's instruction. At the end of study, mice were euthanized. The spleens were excised, photographed, then fixed in 4% paraformaldehyde, paraffin-embedded and stained with hematoxylin and eosin.

### Quantification, Statistical Analysis and Ethics Statement

No pre-processing of data was performed. All the Western blotting, immunofluorescence and RT-qPCR data were obtained from at least three repeated experiments. The data were analyzed using Prism 5.0 software (GraphPad, USA) and are presented as the mean values (standard error of the mean, ±SEMs). Statistical significance between two groups was determined by unpaired two-tailed Student's t test. Multiple-group comparisons were performed using one-way analysis of variance (ANOVA). Differences were considered to be significant for P<0.05 (indicated with an asterisk (*)). This study was approved by the Ethics Committee of Westlake University.

### Example 1: LRP1 was Highly Expressed in Leukemia Patients

Dysregulation of the Notch pathway is frequently observed in congenital heart disease (CHD), one of the most severe genetic disorders. 731 high-confidence candidate interacting proteins (HCIPs) in whole-exome sequencing data from 2871 CHD probands were searched and 253 curated human/mouse CHD genes were searched using a hierarchical approach. NOTCH1, LRP1, CHD7, FBN2, and DYNC2H1 were the most important CHD-related HCIPs.

Similarly, dysregulation of Notch signaling, such as genetic alterations of Notch pathway components, leads to many types of cancer, including T-ALL and SCCs. To explore the correlation between HCIPs and Notch-related cancers, we searched HCIPs in exome sequencing data of 130 T-ALL patients and identified LRP1 as one of the top candidates involved in T-ALL.

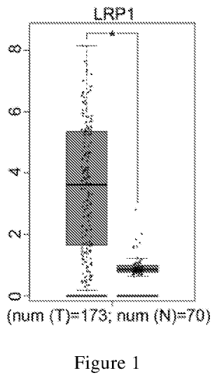

We explored the genes related to leukemia using differential expression analysis and survival analysis. We found that LRP1 was highly expressed in leukemia patients (FIG. 1).

### Example 2: LRP1 Regulates Notch Signaling

The canonical Notch signaling pathway relies on the ligand binding to its receptors, making the modulation of ligand activity a critical step for the precise regulation of Notch signaling activation. Increasing studies have demonstrated the critical role of ligand endocytosis in Notch activation. Therefore, it is essential to unveil the comprehensive regulation of the ligand-dependent Notch pathway, promoting the development of therapeutic targets in Notch-related diseases.

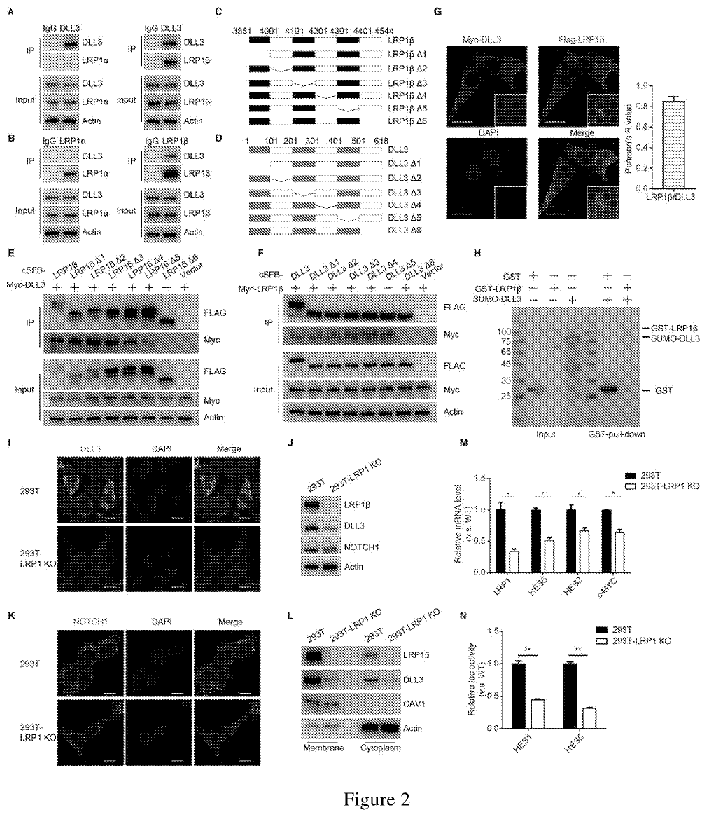

From the MS data, we found that LRP1 interacts with both the Delta and Jagged ligands but not with the Notch receptors. Since LRP1 binds to DLL3 with a good affinity, we chose DLL3 for mechanistic studies. We first validated the interaction between endogenous LRP1 and DLL3 in HEK293T cells (FIGS. 2A and 2B). DLL3 binds to the intracellular subunit LRP1β (FIGS. 2A and 2B). To identify the binding regions on LRP1 and DLL3, we generated a series of truncation mutants for LRP1β and DLL3 (FIGS. 2C and 2D). We found that the C-terminal region (aa 4401-4544) of LRP1 (FIG. 2E) and the C-terminal region (aa 501-618) of DLL3 (FIG. 2F) are responsible for their interaction. LRP1 and DLL3 presumably colocalized on cell membranes and in cytoplasmic vesicles (FIG. 2G). In vitro binding assays further confirmed that LRP1β directly interacts with DLL3 (FIG. 2H).

To investigate whether and how LRP1 regulates Notch signaling, we knocked out LRP1 in HEK293T cells using the CRISPR-Cas9 system and evaluated its impact on DLL3 and NOTCH1. Knocking out LRP1 markedly decreased the protein level as well as the membrane localization of DLL3 (FIGS. 2I, 2J), while the NOTCH1 localization and level were largely unaffected (FIGS. 2J and 2K). We further separated the membrane and cytosolic fractions and found that knocking out LRP1 mainly disturbed DLL3 in the membrane fraction (FIG. 2L). To understand the role of LRP1 in regulating Notch signaling, we detected the impact of LRP1 knockout (KO) on target genes of the Notch pathway. Knocking out LRP1 significantly decreased the mRNA levels of HES2, HES5 and c-MYC (FIG. 2M). A luciferase assay also demonstrated the decreased activity of HES1 and HES5 reporters upon LRP1 KO (FIG. 2N). Together, these findings suggest that LRP1 plays a critical role in Notch signaling, which is mediated by regulating DLL3 membrane localization and protein stability.

### Example 4: Overexpression of LIN-12 Rescues the Molting Defect Induced by lrp-1 RNAi in C. elegans

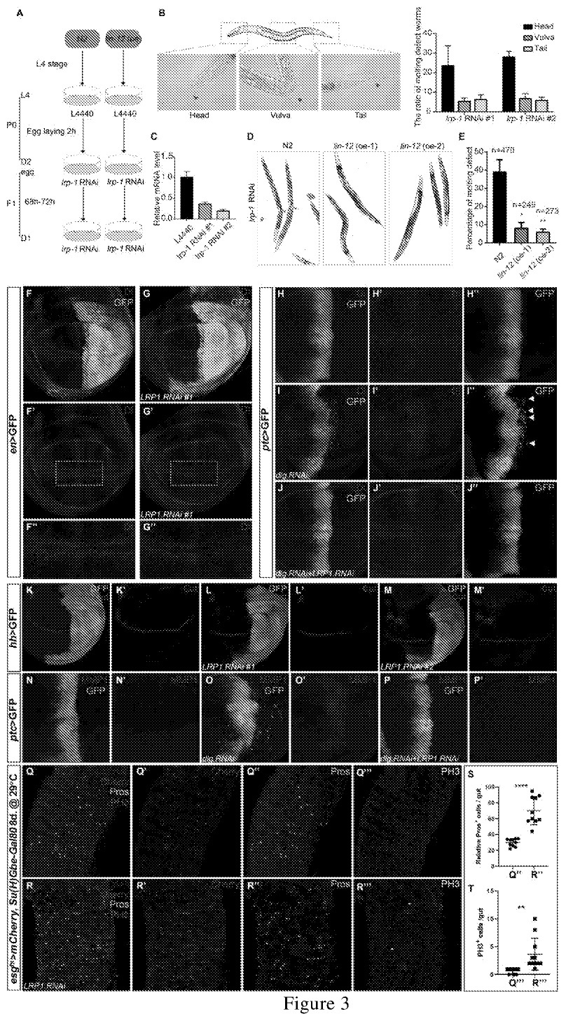

The Notch signaling pathway is highly conserved in various species ranging from C. elegans and Drosophila to mammals. We wondered whether the regulatory mechanism of LRP1 in the Notch pathway is also conserved in other organisms. First, we knocked down the LRP1 levels in C. elegans (FIG. 3A) and we indeed observed a small portion of animals with abnormal vulva phenotypes when lrp-1 is knocked down by RNAi (FIGS. 3B and 3C). However, there are much larger percentages showing molting defects and the bubble-like phenotype occurring randomly in the whole body of lrp-1 RNAi animals (which theoretically includes the bubble-like phenotypes in or around vulva) (FIG. 3B). These are typical phenotypes observed in Notch-disordered nematodes. To further explore the role of LRP-1 in Notch signaling, we constructed two transgenic lines overexpressing the Notch1 orthologue gene lin-12 (FIG. 3A). We found that LIN-12 overexpression reversed the molting defect phenotype caused by lrp-1 RNAi (FIGS. 3D and 3E). These results demonstrate that LRP1 acts upstream of the Notch pathway and positively regulates the Notch pathway in C. elegans.

### Example 5: LRP1 Positively Regulates Delta and Notch Signaling in Drosophila

To further explore the physiological functions of LRP1 and to test whether our findings that LRP1 activates Notch signaling by stabilizing DLL3 are evolutionarily conserved in vivo, we knocked down the Drosophila LRP1 orthologue (LDL receptor protein 1, CG33087) by dsRNA (RNAi) in various organs. Consistent with the role of LRP1 in promoting DLL3 stabilization in mammalian cells, LRP1 reduction in the Drosophila eye imaginal disc or in the posterior region of the wing imaginal disc in lrpl mutant clones or using two independent LRP1 RNAi strains all significantly decreased the level of endogenous Delta (D1), the Drosophila orthologue of DLL3 (FIGS. 3F-G′ and 4A-C″). Additionally, LRP1 knockdown in the posterior region of wing discs reduced endogenous Cut expression, a classic Notch target gene (FIGS. 3K-M′). Downregulation of cell polarity genes, including disc large (dlg), scribble (scrib), and lethal giant larvae (lgl) driven by ptc-Gal4 along the anterior/posterior (A/P) compartment boundary of the wing disc, resulted in cell invasion behavior, in which cells delaminated and migrated towards the posterior part (FIGS. 3N and 3O); these effects were accompanied by increased expression of MMP1, a matrix metalloprotease essential for basement membrane degradation (FIGS. 3N′ and 3O′), as well as upregulation of D1 (FIGS. 3H′ and 3I′). We found that all the phenotypes were dramatically suppressed by RNAi downregulation of LRP1 (FIGS. 3J and 3P), indicating that LRP1 is required for cell polarity loss-induced cell invasion and D1 upregulation.

### Example 6: Knocking Out LRP1 Attenuates Notch Signaling-Dependent Leukemia Invasion, Migration, and Tumorigenesis

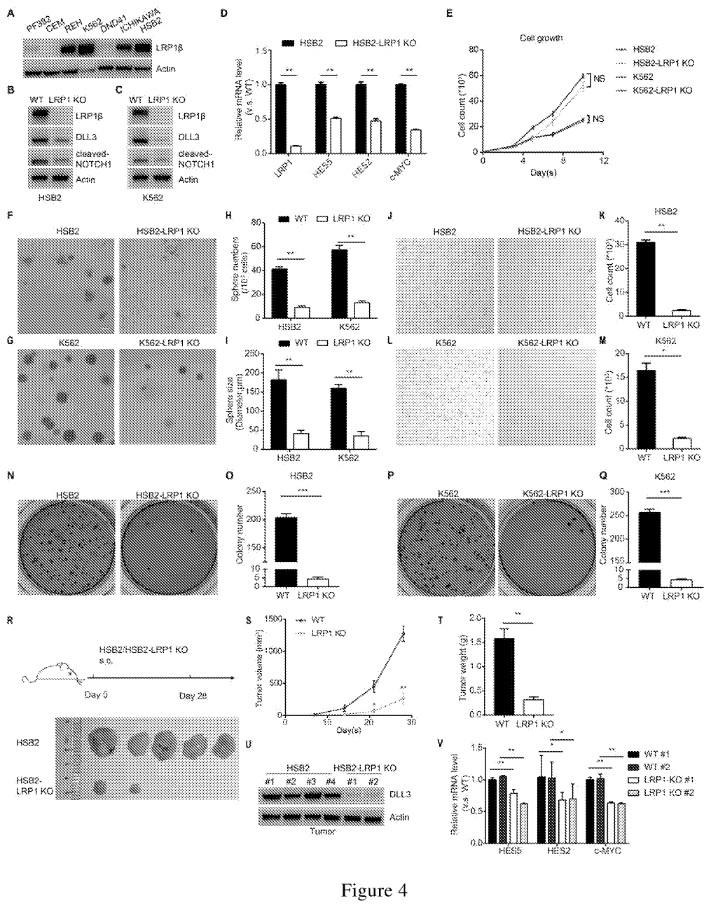

Notch signaling has been linked to multiple types of leukemia, including ALL and acute myeloid leukemia (AML). We wondered whether LRP1 functions in leukemia pathogenesis by regulating the Notch pathway. LRP1 is highly expressed in several leukemia cell lines, including the NOTCH1 wild-type T-ALL cell line HSB2 and the AML cell line K562 (FIG. 4A), as well as leukemia patients (FIG. 1). Knocking out LRP1 in HSB2 and K562 cells reduced the protein levels of DLL3 and cleaved NOTCH1 (FIGS. 4B and 4C) and subsequently attenuated the expression of Notch target genes (FIG. 4D), indicating that LRP1 plays a critical role in Notch signaling activation in leukemia cells.

To further explore the role of LRP1 in leukemia pathogenesis, we knocked out LRP1 in HSB2 and K562 cells and evaluated the impact on leukemia cell proliferation, invasion, migration, and tumorigenesis. Knocking out LRP1 did not prominently interfere with leukemia cell proliferation (FIG. 4E); however, it significantly reduced leukemia cell invasion (FIGS. 4F-I), migration (FIGS. 4J-M), and anchorage-independent cell growth (FIGS. 4N-Q). Moreover, both tumor size and tumor weight in mice injected with HSB2 LRP1-KO cells were significantly reduced compared with those in mice injected with control HSB2 cells (FIG. 4R-T). Western blotting assays confirmed that the protein level of DLL3 was significantly reduced in xenograft tumors formed in HSB2 LRP1-KO cell-injected mice (FIG. 4U), accompanied with decreased mRNA levels of Notch target genes (FIG. 4V). Together, these data suggest that LRP1 plays a key role in leukemia tumorigenesis both in vitro and in vivo.

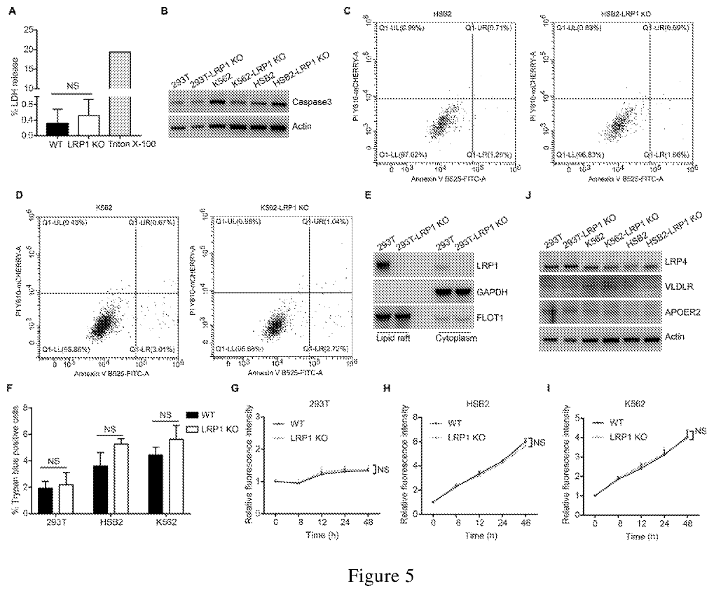

Considering that LRP1 is an endocytic receptor that internalizes a vast number of ligands, including lipoproteins, and could therefore play a critical role in cellular activity. Thus, we next tested whether LRP1-deficient cells were normally viable. Comparing with wild-type cells, no differences in viability and apoptosis were recorded, as measured by LDH release assays (FIG. 5A), Caspase3 activation and Annexin V/PI staining (FIG. 5B-5D). Also, we examined whether the phenomenon observed in LRP1 KO cells were due to the impaired cholesterol uptake considering its role in lipid metabolism. No disruption of lipid raft was observed in LRP1 KO cells as indicated by FLOT1 expression (FIG. 5E) and the membrane integrity as well as cholesterol uptake ability were also not affected by LRP1 deficiency (FIG. 5F-5I). To analyze whether the loss of LRP1 could be compensated for by other lipoprotein receptors, we examined the expression of LRP4, ApoER2 and VLDLR, respectively, in WT and LRP1 KO cells. There was no difference in expression levels between WT and LRP1 KO cells either in 293T or leukemia cells (FIG. 5J), indicating no prominent role for compensation by other lipoprotein receptors in LRP1 deficient cells.

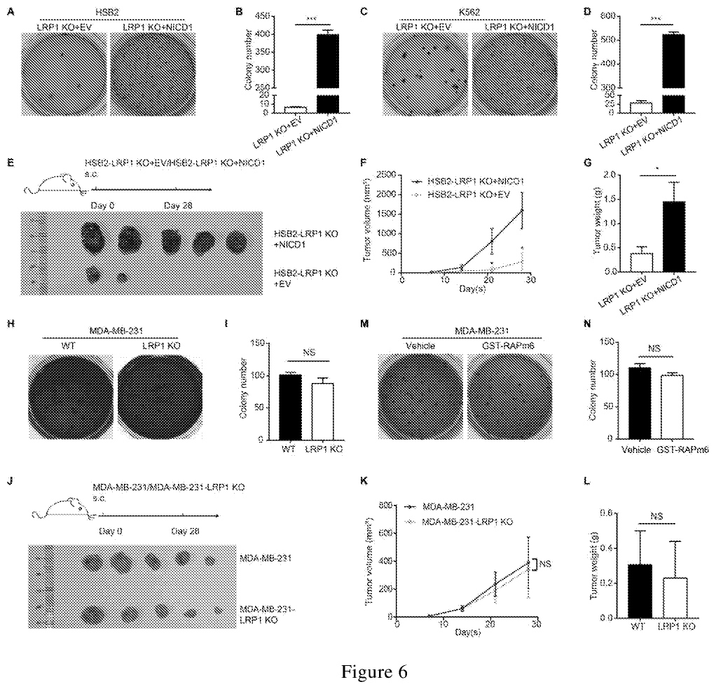

To further verify that the function of LRP1 in leukemia pathogenesis depends on its regulation of Notch signaling, we overexpressed Notch intracellular domain 1 (NICD1), the active form of NOTCH1, in LRP1-KO HSB2 and K562 cell lines and investigated the tumorigenesis ability of these cell lines both in vitro and in vivo. Reconstitution with NICD1 fully rescued colony formation (FIGS. 6A-D) and xenograft tumor growth (FIGS. 6E-G) in LRP1 KO cells. We also knocked out LRP1 in MDA-MB-231 breast cancer cells with low Notch activity. Knocking out LRP1 showed no prominent effect on colony formation (FIGS. 6H and 6I) or xenograft tumor formation (FIGS. 6J-L). Taken together, our data indicate that LRP1 promotes leukemia invasion, migration, and tumorigenesis in a Notch signaling-dependent manner.

### Example 7: LRP1 Antagonist RAPm6 Inhibits Tumorigenesis in Human Leukemia Cells, Mouse Xenografts and Leukemia Models

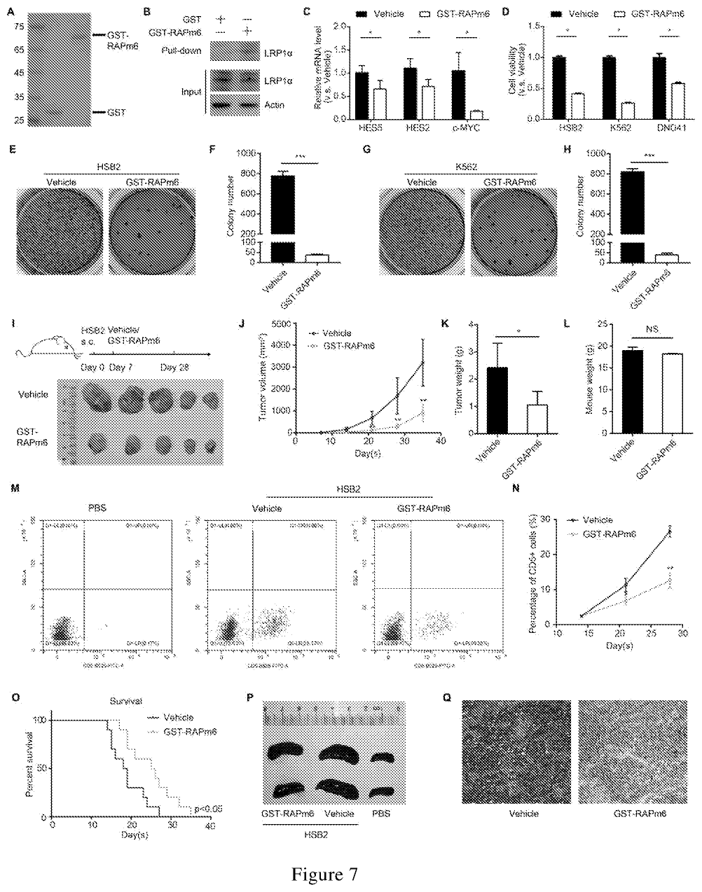

Since LRP1 positively regulates Notch signaling and leukemia tumorigenesis, it may serve as a therapeutic target for Notch signaling-related LRP1-overexpressing cancers. Alpha-2-macroglobulin receptor-associated protein (LRPAP1) is a known LRP1 antagonist that has previously been used to block LRP1-related blood-brain barrier opening in a mouse model. We purified its mutant RAPm6, whose amino acid sequence has been optimized to increase its stability, from Escherichia coli (FIG. 7A) and found that RAPm6 interacts with LRP1α (FIG. 7B). RAPm6 treatment significantly decreased Notch target gene expression (FIG. 7C), the cell viability of several leukemia cell lines (FIG. 7D), anchorage-independent colony formation (FIGS. 7E-H), and xenograft tumor growth (FIGS. 7I-K) without significantly affecting mouse weight (FIG. 7L). RAPm6 treatment did not elicit a prominent effect on xenograft tumor growth of Notch-low MDA-MB-231 cells (FIGS. 6M and 6N), indicating that RAPm6 treatment specifically targets Notch signaling-related cancers.

We also established a leukemia mouse model via tail vein injection of HSB2 cells into NOD-SCID mice. Compared with vehicle treatment, RAPm6 treatment significantly decreased the number of CD5+ HSB2 leukemia cells in the peripheral blood of the mice (FIGS. 7M and 7N). Compared with the vehicle-treated group, the RAPm6-treated group experienced improved mouse survival against leukemia (FIG. 7O). We isolated the spleens and observed apparent splenomegaly in the vehicle group, which was alleviated by RAPm6 injection (FIG. 7P). Histological analysis also confirmed less lymphocytes infiltration into tissues (FIG. 7Q). Together, these results suggest that the LRP1 antagonist RAPm6 alleviates the progression of leukemia.

The present invention is not limited to above embodiments. Any variation, modification, substitution, combination, and simplification without departing from the spirit and principle of the present invention belongs to equivalents of the present invention and is included within the scope of protection of the present invention.

1. A method for treating Notch signaling-dependent disease in the subject with a LRP1 inhibitor.

2. The method of claim 1, wherein the LRP1 inhibitor is a polypeptide antagonist specifically against LRP1, an RNA polynucleotide specific to LRP1, or a small molecule compound inhibitor specific to LRP1.

3. The method of claim 2, wherein polypeptide antagonist is LRPAP1 or LRPAP1 derivative thereof that can bind to LRP1 on the cell surface and prevent ligands from its binding.

4. The method of claim 3, wherein the LRPAP1 is a polypeptide comprising:
1) an amino acid sequence of SEQ ID NO:1 or 2;
2) an amino acid sequence at least about 70%, about 80%, about 85%, about 90%, about 95%, about 99%, or more identity to SEQ ID NO:1 or 2; or
3) an amino acid sequence with addition, deletion and/or substitution of one or more amino acids compared with SEQ ID NO:1 or 2,
the LRPAP1 can bind to LRP1 on the cell surface, preventing ligands from its binding.

5. The method of claim 3, wherein the LRPAP1 derivative is a polypeptide comprising:
1) an amino acid sequence of SEQ ID NO:3;
2) an amino acid sequence an amino acid sequence with at least about 70%, about 80%, about 85%, about 90%, about 95%, about 99%, or more identity to SEQ ID NO:3, or
3) an amino acid sequence with addition, deletion and/or substitution of one or more amino acids compared with SEQ ID NO:3,
the LRPAP1 derivatives can bind to LRP1 on the cell surface, preventing ligands from its binding.

6. The method of claim 3, wherein the LRPAP1 derivative is a polypeptide comprising:
an amino acid sequence of SEQ ID NO:4 or an amino acid sequence with at least about 70%, about 80%, about 85%, about 90%, about 95%, about 99%, or more identity to SEQ ID NO:4, or an amino acid sequence with addition, deletion and/or substitution of one or more amino acids compared with SEQ ID NO:4.

7. The method of claim 6, wherein the LRPAP1 derivative is a polypeptide comprising SEQ ID NO: 4

8. The method of any one of claim 4-6, wherein the LRPAP1 or LRPAP1 derivative is a polypeptide with or without a tag.

9. The method of claim 8, wherein the tag is selected from c-Myc, His, HA, GST, MBP, Flag, and Arg6.

10. The method of any one of claim 4-6, wherein the LRPAP1 or LRPAP1 derivative is a polypeptide modified by PEG.

11. The method of claim 2, wherein polypeptide antagonist is an antibody against LRP1.

12. The method of claim 2, wherein the RNA polynucleotide is selected from siRNA, shRNA, guide RNA, and miRNA.

13. The method of claim 9, wherein the guide RNA is SEQ ID NO: 5 (TGGAGGACAAGATCTACCGC).

14. The method of claim 1, wherein the Notch signaling-dependent disease is selected from leukemia, myeloma, lymphoma, breast cancer, liver cancer, and lung cancer.

15. The method of claim 14, wherein the leukemia is T-acute lymphoblastic leukemia or Chronic lymphocytic leukemia.

16. The method of claim 1, wherein the subject is non-human mammal or human.

17. The method of claim 1, wherein the disease is a metastatic cancer.

18. A method of screening medicines for treating Notch signaling-dependent disease using LRP1 as the target, the method comprising: observing the effect of candidate medicine on the expression or activity level of LRP1, if the candidate medicine can inhibit expression or activity level of LRP1, then it indicates that the candidate medicine is a potential medicine for treating Notch signaling-dependent disease.

19. The method of claim 18, wherein the Notch signaling-dependent disease is selected from leukemia, myeloma, lymphoma, breast cancer, liver cancer, and lung cancer.

20. The method of claim 19, wherein leukemia is T-acute lymphoblastic leukemia or Chronic lymphocytic leukemia.
